(19) United States
(12) Patent Application Publication
(10) Pub. No.: US 2005/0244397 A1
(43) Pub. Date: Nov. 3, 2005

(54) Process for prolonging the after-harvest life of citrus fruit

(76) Inventor: Stephen J. Gorton, Rowlett, TX (US)

Correspondence Address: David S. Taylor, Esq.;Berenato, White & Stavish, LLC, Suite 240, 6550 Rock Spring Drive, Bethesda, MD, 20817, US

(21) Appl. No.: 11/179,645

(22) Filed: Jul. 13, 2005

Related U.S. Application Data
(63) Continuation of application No. 10/688,671, filed on Oct. 20, 2003.

(51) Int. Cl. A61K 38/54

(52) U.S. Cl. 424/94.2

(57) ABSTRACT

A formulation is provided for preventing fungus on citrus fruits to prolong the life of the fruits after harvesting. The formulation is based on a combination of safe organic enzymes and catalysts by utilizing natural microbal action to break down carbohydrate forming cell walls by consuming and digesting the offending fungus spores, mycotoxins, bacteria and other biological pathogens on contact. The formulation may be either atomized in vivo or on the fruit or by a drenching method.

### CROSS REFERENCE TO RELATED APPLICATIONS

This is a continuation of U.S. patent application Ser. No. 10/688,671 filed on Oct. 20, 2003, the complete disclosure of which is incorporated herein by reference.

### STATEMENT REGARDING FED SPONSORED R&D

(none)

### BACKGROUND OF THE INVENTION

In the process of prolonging the after-harvest life of citrus fruits it is important that the life of the fruits be prolonged or extended because the fruits are only grown in certain areas of the globe and must be shipped by various carries, different modes of transportation and through different climates. When not treated in various ways and while in transit or on store shelves or even after purchase by a consumer, the citrus fruits can and will develop various damaging proteins on their outer surfaces which will rapidly destroy the usefulness of the fruits. Because this is so, the citrus producing companies as well as the countries that support this industry are experiencing tremendous losses. It would be of a considerable advantage and help if these damaging influences could be eliminated or at least curtailed.

The offending fungus of the above noted problems have been identified by various laboratories throughout the country including the “Florida Department of Citrus”. The Florida Dept. of Citrus is especially interested in this endeavor because the State of Florida is a major producer of citrus fruits and does not ship citrus fruit only within all of the states of the USA but is heavily involved in shipping fruit to many offshore countries. As mentioned above, any losses during shipping alone constitute a heavy financial burden not only on growers, producers, shippers as well as State revenue. Awareness of the fungus problem, with an endless succession of media articles and feature stories detailing human health hazards, homes, schools and workplaces unfit for occupancy, downtime and lost productivity.

The traditional remedies that have been used are chemical bleach washes, and VOC's demolition and/or reconstruction projects including tossing out large amounts of rotten fruits. These projects have been more and more discredited as largely ineffective and unnecessarily costly and time consuming. Innovation is inevitable under such circumstances. This innovation will be explained below.

### SUMMARY OF THE INVENTION

The offending fungus has been identified as various spores, mycotoxins, bacteria and other biological pathogens. The inventive formula was formulated exclusively to combat fungus and dangerous mycotoxins. Safety and efficacy has been documented by laboratory studies and field applications across the country.

### DETAILED DESCRIPTION OF THE INVENTION

The inventive formula of the invention consists of a combination of safe organic enzymes and catalysts utilizing natural microbal action to break down carbohydrate-forming cell walls, consuming and digesting the offending fungus spores, mycrotoxins, bacteria and other biological pathogens on contact, completely penetrating all porous surfaces and leaving behind an effective surfactant against regrowth which is good for one year. The formulation continuos to work until all contiguous nutrient sources have been eliminated. The formulation used in the following process is a non-toxic enzyme formulation that neutralizes proteins and removes stains.

The method of prolonging the after-harvest life of the citrus fruits then is to simply spray the fruits with the formula explained above. However, the spraying step may involve a two step process:
1) liquid spray and
2) atomization.

The liquid spray may involve a drenching of the fruits.

(1) The atomization system may involve a misting of the formula of particles under 7 microns.

The atomization process is preferred because the misting substance becomes airborne in a storage room before the fungus has a chance to grow on the surface of the fruit.

(2) The drench process involves putting the fruits into a water base wax in a container and then removing the fruits from the container.

In either of the two above mentioned processes, it has been found that the shelf life or after-harvest life of the fruit has been extended or prolonged to a considerable extent that most likely will enhance the profit margin of the grower, the producer and the state revenue to a considerable extent.

The enzyme-based formula of the present invention protects the plant workers against carciginous effects because the inventive product is a non-chemical and non-synthetic product that does not have any known side effects.

1. A process for prolonging an onset of a fungus invasion on citrus fruits from after harvesting to delivery to a consumer, comprising treating a multiple of the citrus fruits with a liquid formulation comprising enzymes and catalysts, and enzymatically breaking down carbohydrate cell walls of fungus on the citrus fruit.

2. The process of claim 1 wherein said treating of the citrus fruits is performed in vivo.

3. The process of claim 1, wherein said treating comprises atomizing the liquid formulation into particles of less than 7 microns.

4. The process of claim 1 wherein said treating comprises drenching of the citrus fruits in a liquid wax based substance, the liquid wax based substance comprising the liquid formulation.

5. The process of claim 1 further comprising washing the citrus fruits at a point of consumer delivery to present a fresh and clean fruit to the consumer.
